(19) United States
(12) Patent Application Publication
(10) Pub. No.: US 2022/0356534 A1
(43) Pub. Date: Nov. 10, 2022

(54) METHOD

(71) Applicant: Givaudan SA, VERNIER (CH)

(72) Inventors: Catherine ZANCHETTA, Toulouse (FR); Cyrille JARRIN, Muret (FR); David VILANOVA, Escaldes (AD)

(21) Appl. No.: 17/641,331

(22) PCT Filed: Sep. 11, 2020
(86) PCT No.: PCT/EP2020/075530

§ 371 Date: Mar. 8, 2022

(30) Foreign Application Priority Data
Sep. 27, 2019 (GB) 1913970.8

(51) Int. Cl. C12Q 1/6888; C12Q 1/6806; C12N 15/10

(52) U.S. Cl. CPC C12Q 1/6888; C12Q 1/6806; C12N 15/1065; C12Q 1/6869

(57) ABSTRACT

The present invention provides a fast and simple method for preparing a microbial profile of a human skin sample. The method can be performed using only portable devices, allowing for in-field profiling. The method is robust and has been found to work even with low DNA quantities and under difficult conditions.

The invention relates to a method for preparing a microbial profile of a human skin sample.

The skin microbiota represents the living microorganisms on the surface of the skin.

In the wake of numerous studies devoted to the intestinal microbiota and demonstration of its major contribution to human health, study of the cutaneous or skin microbiota has grown considerably over the last years. Curing skin diseases such as acne, atopic dermatitis or psoriasis has long been a driving force in investigating the skin microbiota. Today, awareness of the significance of the skin microbiota greatly exceeds the sphere of pathogenic skin microbial ecology, and understanding of the complex interactions between healthy human skin and the to microorganisms inhabiting it promises major developments in the field of dermocosmetics. Thus, there is an increasing interest in cosmetic products that are able to preserve the natural microbial balance of healthy and beautiful skin.

Bacteria, fungi, viruses and archaea are the main microorganisms constituting the microbiota, which is defined as the microbial communities inhabiting a specific environment. In humans, many efforts have been made to characterize the different body site ecosystems and their associated microbial communities, mainly at bacterial level, which are the most abundant microorganisms on the human-associated microbiota.

There are two fundamental method types for identifying these microorganisms and getting a representation of the composition of the microbiome: Culture-based methods and culture-free methods.

Culture-based methods can provide an identification at the species level using morphology and enzymatic activities identification. This approach is restricted to cultivable microorganisms and involves the growth of bacteria on culture media. Consequently, the time for identification is limited by the time necessary for the bacterial growth.

There are several different culture-free methods: Polymerase Chain Reaction (PCR), MALDI-TOF MS and sequencing. The speed of culture-free analysis is limited by the time necessary for DNA extraction and low DNA quantities. PCR can provide an identification at the strain level, but is limited to pre-defined bacteria targeted by the user. MALDI-TOF MS and sequencing of bacterial genomes are both based on the acquisition of data regarding the entire microbiota profile. These data are then compared to databases.

Biomolecule extraction, such as deoxyribonucleic acid (DNA) and ribonucleic acid (RNA) from a variety of starting biological materials to be used in downstream applications and other analytical or preparative purposes, is the most important first step in molecular biology. Four indispensable steps are generally required for successful nucleic acid purification:
1. Cell lysis through disruption of the cellular membranes, cyst wall or egg wall;
2. dehydration and precipitation of the cellular proteins (protein denaturation);
3. separation of cellular proteins and other cellular components out of the nucleic acid; and
4. precipitation and dissolving the nucleic acid.

The most common strategy to assess bacterial microbiota is amplifying and sequencing specific regions of 16S rRNA gene using 2^{nd} generation massive sequencing technologies. This bacterial marker gene is ubiquitously found in bacteria, and has nine hypervariable regions (V1-V9) that can be used to infer taxonomy.

The ability to classify sequences to the genus or species level is a function of read length, sample type, the reference database, and the quality of the sequence. High-quality short-reads obtained from 2^{nd} generation sequencers (250-350 bp) bias and limit the taxonomic resolution of this gene. The most common region amplified with Illumina MiSeq or Ion Torrent PGM™ for bacterial taxonomic classification is V4, but this region fails to amplify some significant species for skin microbiota studies, such as Propionibacterium acnes. An alternative choice when performing a skin microbiota study is the V1-V2 regions, although they lack sensitivity for the genus Bifidobacterium and poorly amplify the phylum Verrucomicrobia. On the other hand, near full-length 16S rRNA gene sequences are required for accurate richness estimations especially at higher taxa, which are necessary on microbiota studies. Besides, full-length reference sequences are needed for performing phylogenetic analyses or designing lineage specific primers, especially in species different to human or mouse, in which previous metagenomics approaches deciphered the richness of bacterial species in the great and different variety of microbiome samples analyzed.

Jarrin et al. have described a metagenomics study performed on human skin samples collected by swabbing in the forearm area, aimed at characterizing the bacterial diversity and measuring the level of inter-individual variability (“Dry Skin Associated Microbiota Diversity Revealed by High-Depth Next Generation Sequencing”; IFSCC Magazine 4, 2017). 16S rRNA sequencing was performed using the MiSeq device (Illumina, Inc., San Diego, Calif., USA), targeting the V3V4 16S variable regions.

With the launching of 3^{rd} generation single-molecule technology sequencers, these short-length associated problems can be overcome by sequencing the full or almost full-length of 16S rRNA gene with different sets of universal primers.

MinION™ sequencer of Oxford Nanopore Technologies (ONT) (https://nanoporetech.com) is a 3^{rd} generation sequencer that is portable, affordable with a small budget and offers long-read output (only limited by DNA extraction protocol). Besides, it can provide a rapid real-time and on-demand analysis very useful on clinical applications. The use of MinION™ to characterize dog skin microbiota has been described by Cusco et al. (“Using MinION™ to characterize dog skin microbiota through full-length 16S rRNA gene sequencing approach”; bioRxiv preprint first posted online Jul. 21, 2017; doi: http://dx.doi.org/10.1101/167015).

Known methods for characterizing human skin microbiota have several drawbacks: They are very time consuming and typically require several days from sampling to microbial profile; and they require heavy equipment and laboratory devices.

It is therefore a problem of the present invention to provide a method for preparing a microbial profile of a human skin sample that is fast, robust, simple, reproducible, and can be performed using only portable devices.

This problem is solved by the method of the present invention described below.

### SUMMARY OF THE INVENTION

The present invention provides a method for preparing a microbial profile of a human skin sample, comprising the steps of:
(a) extracting DNA from the human skin sample to obtain a crude DNA extract;
(b) treating the crude DNA extract with RNase to remove RNA and obtain an RNA-depleted DNA extract;
(c) purifying the RNA-depleted DNA extract to remove small DNA fragments to obtain a first concentrated DNA extract;
(d) amplifying the DNA of the first concentrated DNA extract to obtain an amplified DNA extract;
(e) purifying the amplified DNA extract to remove small DNA fragments to obtain a second concentrated DNA extract;
(f) preparing a DNA library from the second concentrated DNA extract;
(g) sequencing the DNA of the DNA library to obtain raw DNA sequences;
(h) removing human DNA sequences from the raw DNA sequences to obtain non-human DNA sequences; and
(i) analysing non-human DNA sequences to obtain the microbial profile.

In one embodiment, the method for preparing a microbial profile of a human skin sample (or method of the invention) some steps are optional or even not necessary, for example when the “16s RNA gene sequencing approach” is used and the sequencing is selected from 16S rDNA sequencing (also named as 16s ribosomal RNA gene sequencing)

“16s RNA gene sequencing approach” means that the gene of the 16s rRNA of the different species of bacteria from the sample of step a) will be amplified and sequenced as described in the method of the invention and in the present description. Both, the full length 16s rRNA gene or some portions of the 16s RNA gene (such as the V1-V3 region) may be analyzed (amplified and sequenced).

Thus, the present invention provides a method for preparing a microbial profile of a human skin sample, comprising the steps of:
(a) extracting DNA from the human skin sample to obtain a crude DNA extract;
(b) optionally treating the crude DNA extract with RNase to remove RNA and obtain an RNA-depleted DNA extract;
(c) optionally purifying the DNA of step a) or the RNA-depleted DNA extract of steps b) to remove small DNA fragments to obtain a first concentrated DNA extract;
(d) optionally amplifying the DNA of the first concentrated DNA extract to obtain an amplified DNA extract;
(e) optionally purifying the amplified DNA extract of step d) to remove small DNA fragments to obtain a second concentrated DNA extract;
(f) preparing a DNA library from the concentrated DNA extract of step c) or from the second concentrated DNA extract from step e);
(g) sequencing the DNA of the DNA library to obtain raw DNA sequences;
(h) optionally removing human DNA sequences from the raw DNA sequences to obtain non-human DNA sequences; and
(i) analysing non-human DNA sequences to obtain the microbial profile.

The method of the present invention allows for preparing a microbial profile of a human skin sample in less than seven hours from sampling to completion. This is essentially a real-time profiling, and is significantly shorter than previous methods.

In addition, the method of the present invention can be performed using only portable devices, allowing for in-field profiling.

The method is robust and has been found to work even with low DNA quantities—skin samples typically have a much lower DNA content than, for example, gut samples (skin: about 0.2 ng/μl; gut: about 25 ng/μl)—and after shaving and/or the application of after-shave, cream and/or make-up.

### DETAILED DESCRIPTION OF THE INVENTION

The method of the present invention may be performed on human skin samples obtained from any desired area of the body, not only from the face, but also from the scalp, axilla or forearm.

The human skin sample may be obtained by any suitable method, for instance by swabbing, scrubbing or scraping. Non-invasive sampling methods are generally preferred.

In an embodiment, the human skin sample is obtained by swabbing. For instance, swabbing may be performed using sterile swabs (e.g. Medicomp, Hartmann; or forensic swabs from Sarstadt; swabs may be made of viscose, optionally also containing some polyester, e.g. 100% viscose or 70% viscose +30% polyester), which are preferably moistened with physiological serum (e.g. serum physiologique, Mercurochrome), with a sterile NaCl solution (e.g. 0.15 M NaCl), or mixtures thereof. Moisturizing the swab prior to swabbing helps to avoid abrasion of the skin.

The DNA extraction of step (a) may be performed by any suitable method. It may be performed directly on the human skin samples obtained by, for instance, swabbing, scrubbing or scraping. Alternatively, said samples may be pre-treated prior to DNA extraction.

In an embodiment, the DNA extraction of step (a) is performed using the DNeasy PowerLyser PowerSoil Kit by Qiagen. The the DNeasy PowerLyser PowerSoil Kit contains MB Spin Columns comprising a white filter membrane, Solutions C1, C2, C3, C4, C5, and C6, a PowerBead Solution, as well as several PowerBead Tubes and Collection Tubes.

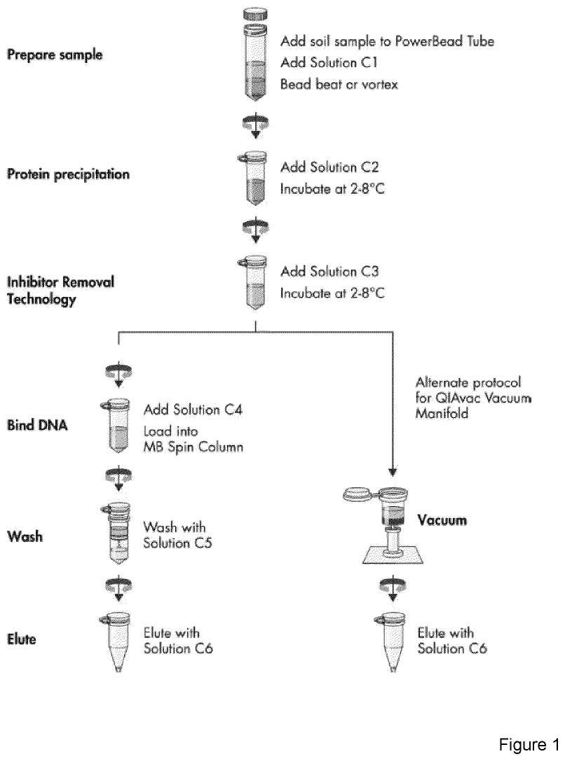

The DNA extraction may be done according to the manufacturer's instructions, e.g. according to the “Experienced User” protocol. The standard procedure is illustrated in FIG. 1.

Preferably, however, a modified procedure as outlined below is used in order to optimize speed and avoiding the use of an incubator.

Therefore, in an embodiment of the present invention, the DNA extraction of step (a) is performed using the DNeasy PowerLyser PowerSoil Kit by Qiagen with a modified procedure, said modified procedure comprising the following steps:
(a1) treating the human skin sample with the PowerBead Solution to obtain a solute sample;
(a2) treating the solute sample with Solution C1, vortexing, and incubating;
(a3) vortexing;
(a4) centrifuging to obtain a first supematant and a first precipitate;
(a5) mixing Solution C2 with Solution C3, adding this mixture to the first supernatant, vortexing, and incubating;
(a6) centrifuging to obtain a second supernatant and a second precipitate;
(a7) adding Solution C4 to the second supernatant and vortexing to obtain a treated supernatant;
(a8) loading a first part of the treated supernatant onto an MB Spin Column, centrifuging, and discarding the flow-through;
(a9) loading a second part of the treated supernatant onto the MB Spin Column, centrifuging, and discarding the flow-through;
(a10) loading the remaining treated supernatant onto the MB Spin Column, centrifuging, and discarding the flow-through;
(a11) adding Solution C5 and centrifuging;
(a12) discarding the flow-through and centrifuging the remainder;
(a13) adding Solution C6 to the MB Spin Column; and
(a14) centrifuging and discarding the MB Spin Column to obtain the crude DNA extract.

Step (a1) may be performed in a PowerBead Tube. According to the manufacturer's instructions, 750 μl of the PowerBead Solution are added to the human skin sample. Alternatively, if the human skin sample is obtained by swabbing, 800 μl of the PowerBead Solution may be added to the swab to obtain a solute sample, and 750 μl of the solute sample may then be used in step (a2).

Step (a2) is an additional step not described in the manufacturer's instructions. This step may also be performed in a PowerBead Tube, preferably the one from step (a1). Typically, 750 μl of the solute sample are treated with 60 μl of Solution C1. Vortexing can be done for any suitable amount of time, for instance for 1-10 s, preferably for 2-5 s, e.g. for 3 s. Incubation is preferably done at room temperature, for instance at a temperature of about 10-30° C., preferably at about 15-25° C., e.g. at about 20-22° C. Incubation time is preferably chosen based on the temperature, for instance about 10 min at 20-22° C.

Vortexing in step (a3) may be done in a single run, or in several repetitions. Typically, a total vortexing time of less than 5 min is enough. For instance, the mixture may be vortexed four times for 45 s each with 15 s break between vortexing. Alternatively, a homogenizer may be used instead of vortex.

In step (a4), a centrifugation time of less than 1 min is sufficient. For instance, centrifuging may be done for 30 s at 10′000 ×g. The first precipitate is removed and the procedure is continued with the first supematant.

Step (a5) is a modification to the protocol described in the manufacturer's instructions. It allows for applying Solutions C2 and C3 together, thereby avoiding additional steps and saving time. To this end, equal amounts of Solution C2 and C3 are mixed. This mixture is then added to the same volume of the first supernatant. For instance, 250 μl of Solution C2 may be mixed with 250 μl of Solution C3, and this mixture then added to 500 μl of the first supernatant. Vortexing can be done for any suitable amount of time, for instance for 1-10 s, preferably for 2-5 s, e.g. for 3 s. Incubation is preferably done at room temperature, for instance at a temperature of about 10-30° C., preferably at about 15-25° C., e.g. at about 20-22° C. Incubation time is preferably chosen based on the temperature, for instance about 5 min at 20-22° C. Step (a5) may be done in a Collection Tube, for instance.

In step (a6), a centrifugation time of less than 1 min is sufficient. For instance, centrifuging may be done for 60 s at 10′000 ×g. The second precipitate is removed and the procedure is continued with the second supematant.

Steps (a7) to (a14) essentially correspond to the steps described in the manufacturer's instructions.

Step (a7) may be done in a Collection Tube, for instance. The Solution C4 is preferably added to the second supernatant in a ratio of about 12:5 v/v, e.g. 1200 μl of Solution C4 may be added to 500μl of the second supernatant. Vortexing can be done for any suitable amount of time, for instance for 1-10 s, e.g. for 5 s.

In steps (a8), (a9) and (a10), three parts of the treated supernatant are consecutively loaded onto the MB Spin Column and centrifuged. At the end of each step, the flow-through is discarded. Preferably, equal amounts of treated supernatant are used in steps (a8) and (a9), and the remainder of the treated supernatant is then used in step (a10), but the parts may also be of other proportions. For instance, 675 μl of the treated supernatant may be used in steps (a8) and (a9). Centrifuging may be done at 10′000 ×g for 60 s, for instance.

In step (a11), Solution C5 is added to the MB Spin Column. Preferably, 500 μl of Solution C5 are added. Centrifuging may be done at 10′000 ×g for 30 s, for instance.

Centrifuging in step (a12) may be done at 10′000 ×g for 60 s, for instance.

Preferably, the MB Spin Column is then moved to a clean tube, e.g. a Collection Tube.

In step (a13), the Solution C6 is preferably applied to the center of the white filter membrane of the MB Spin Column. Preferably, 100 μl of Solution C6 are added. Alternatively, sterile DNA-free PCR-grade water or TE buffer may be used instead of Solution C6.

Centrifuging in step (a14) may be done at 10′000 ×g for 30 s, for instance.

The thus obtained crude DNA extract (typically about 100 μl, depending on the amount of Solution C6 added in step (a13)), may be directly used in the next step (b) of the method of the present invention.

Step b) may be optional.

In one embodiment of the method of the invention the whole genome sequencing approach is used (using for example next generation sequencing, Sanger-sequencing, etc)

In step (b) of the method of the present invention, the crude DNA extract from step (a) is treated with RNase to remove RNA and obtain an RNA-depleted DNA extract. This is an additional step that is commonly not performed in the method of the state of the art. Step (b) allows for improving the DNA by removing RNA.

In an embodiment of the present invention, the crude DNA extract is treated with an aqueous RNAse solution and then incubated.

The aqueous RNAse solution is typically prepared using ultra-pure water. Any suitable concentration may be used, for instance a concentration of 0.1 to 10 mg/ml, preferably 0.5 to 2 mg/ml, most preferably of about 1 mg/ml. The ratio between the crude DNA extract and the aqueous RNAse solution is preferably chosed based on the concentration of the aqueous RNAse solution. For instance, for an aqueous RNAse solution having a concentration of 1 mg/ml, a ratio of the crude DNA extract to the aqueous RNAse solution of 100:6 v/v may be used.

The mixture of the crude DNA extract and the aqueous RNAse solution may be incubated at a temperature of about 10-70° C., preferably of about 20-50° C., more preferably of about 30-40° C., for instance at 37° C. The incubation time is advantageously chosen depending on the incubation temperature. For instance, the mixture may be incubated at 37° C. for 15 min.

In another embodiment, 16s RNA gene sequencing approach is used (using for example next generation sequencing, Sanger-sequencing, etc) and step b) is optional, and thus the DNA extracted in step a) can be directly purified (in step c) to obtain a concentrated DNA extract.

Purification of the RNA-depleted DNA extract in step (c) may be achieved through any suitable method. The main aim of this step is to remove short DNA fragments, which are usually difficult to assign, from the sample, thereby also concentrating the DNA sample to facilitate subsequent steps. This step of purification can be also optional when using the 16s RNA gene sequencing approach. However in a preferred embodiment this step is perform since it improves sequencing results obtained in the next steps.

In an embodiment of the present invention, the RNA-depleted DNA extract is purified using magnetic beads. The use of magnetic beads for size selection within DNA samples is well-known, and there are several commercial suppliers providing such beads (e.g. CleanPCR from CleanNA; NucleoMag NGS Clean-up and Size Select from Macherey-Nagel; or Magnetic Beads PCR Cleanup Kit from Geneaid).

Depending on the ratio of DNA sample to magnetic beads, a different cut-off size may be determined. Upon treatment with the magnetic beads, larger DNA fragments are trapped in the beads, whereas smaller DNA fragments stay in the supernatant. By separating the two using an (external) magnet and, respectively, concentration of the supernatant or washing of the beads (e.g. with 70% ethanol) followed by elution, the smaller or larger DNA fragments may be retrieved, respectively. It is also possible to perform two purification cycles to obtain both an upper and lower cut-off size, if desired.

In the present invention, DNA fragments having a base pair length of less than 500 bp, more preferably of less than 700 bp, and most preferably of less than 1000 bp are removed from the RNA-depleted DNA extract in order to obtain the first concentrated DNA extract.

In an embodiment of the present invention, the purification is a 0.4× purification. This will remove DNA fragments having a base pair length of less than about 1000 bp.

For example, a 0.4× purification may be performed, using e.g. CleanPCR magnetic beads from CleanNA (http://www.cleanna.com/cleanngs/). This will provide an optimal sample for the further method steps. For instance, 106 μl of the RNA-depleted DNA extract may be treated with 42.4 μl of the magnetic beads, corresponding to a ratio of RNA-depleted DNA extract to magnetic beads of 10:4 v/v. The mixture may then left for some time, e.g. for a few minutes, for instance for 8 min, to allow the DNA fragments to link to the magnetic beads.

After application of a magnet, e.g. using a magnetic rack, and removal of the supernatant, the magnetic beads may be rinsed with 70% ethanol in water in order to remove remaining buffer and other residues, and the DNA fragments are then eluted using 5 to 40 μl of an elution buffer, more preferably 5 to 20 μl, for instance about 10 μl, to obtain the first concentrated DNA extract. A suitable elution buffer is, for instance, 10 mM Tris-Cl at a pH of 8.5. The magnetic beads may be left in the elution buffer for a certain amount of time in order to allow full retrieval of the DNA fragments from the beads, for instance for a few minutes, e.g. for 10 min. During this time, the mixture may be inverted or stirred. It is also possible to heat the mixture, but room temperature does also work. For example, the mixture may be inverted and then left for 10 min at 20-22° C.

The magnetic beads may then be removed using the magnet.

The first concentrated DNA extract thus obtained may be used directly in the next steps. Alternatively, it may be further diluted or treated, if desired.

In an embodiment of the present invention, the DNA content of the first concentrated DNA extract is determined by DNA quantification prior to step (d). This allows for a fine-tuning of the amounts and reagents used in the subsequent steps. DNA quantification may be achieved using a Qubit High Sensity Kit (e.g. Qubit dsDNA HS Assay kit, Life technologies; catalog nos Q32851, Q32854) and a Qubit Fluorometer (e.g. from Thermo Fisher Scientific or Invitrogen), but any other suitable devices may also be used.

The method of the invention comprises a step d) of amplifying the DNA of the first concentrated DNA extract to obtain an amplified DNA extract.

In step (d) of the method of the present invention, the DNA of the first concentrated DNA extract is amplified in order to increase the DNA quantity, providing an amplified DNA extract. This step is not part of the conventional methods previously known. Thanks to this amplification step, the DNA quantity is significantly increased, thereby allowing the use of less sensitive detection methods, in particular for DNA sequencing. More particularly, this allows for the use of a field sequencing kit in step (g) of the method of the present invention.

In an embodiment of the present invention, the DNA amplification of step (d) is a Whole Genome Amplification (WGA). Preferably, the DNA amplification, and in particular the Whole Genome Amplification, is achieved by Multiple Displacement Amplification (MDA). One particularly suitable device for this step is the REPLI-g® Advanced DNA Single Cell Kit from QIAGEN (https://www.qiagen.com/us/products/next-aeneration-sequencing/single-cell-low-input/repli-g/repli-g-advanced-dna-sinale-cell-kit/#productdetails), which may be used according to the manufacturer's instructions.

The amplified DNA extract thus obtained may be used directly in the next steps. Alternatively, it may be further diluted or treated, if desired.

Purification of the amplified DNA extract in step (e) may be achieved through any suitable method. The main aim of this step is to remove short DNA fragments, which are usually difficult to assign, from the sample, thereby also concentrating the DNA sample to facilitate subsequent steps.

In an embodiment of the present invention, the amplified DNA extract is purified using magnetic beads. The use of magnetic beads for size selection within DNA samples is well-known, and there are several commercial suppliers providing such beads (e.g. CleanPCR from CleanNA; NucleoMag NGS Clean-up and Size Select from Macherey-Nagel; or Magnetic Beads PCR Cleanup Kit from Geneaid).

Depending on the ratio of DNA sample to magnetic beads, a different cut-off size may be determined. Upon treatment with the magnetic beads, larger DNA fragments are trapped in the beads, whereas smaller DNA fragments stay in the supernatant. By separating the two using an (external) magnet and, respectively, concentration of the supernatant or washing of the beads (e.g. with 70% ethanol) followed by elution, the smaller or larger DNA fragments may be retrieved, respectively. It is also possible to perform two purification cycles to obtain both an upper and lower cut-off size, if desired.

In the present invention, DNA fragments having a base pair length of less than 500 bp, more preferably of less than 700 bp, and most preferably of less than 1000 bp are removed from the amplified DNA extract in order to obtain the second concentrated DNA extract.

In an embodiment of the present invention, the purification is a 0.4× purification. This will remove DNA fragments having a base pair length of less than about 1000 bp.

For example, a 0.4× purification may be performed, using e.g. Clean PCR magnetic beads from CleanNA (http://www.cleanna.com/cleanngs/). This will provide an optimal sample for the further method steps. For instance, 106 μl of the amplified DNA extract may be treated with 42.4 μl of the magnetic beads, corresponding to a ratio of amplified DNA extract to magnetic beads of 10:4 v/v. The mixture may then left for some time, e.g. for a few minutes, for instance for 8 min, to allow the DNA fragments to link to the magnetic beads.

After application of a magnet, e.g. using a magnetic rack, and removal of the supernatant, the magnetic beads may be rinsed with 70% ethanol in water in order to remove remaining buffer and other residues, and the DNA fragments are then eluted using 5 to 40 μl of an elution buffer, more preferably 10 to 20 μl, for instance about 15 μl, to obtain the first concentrated DNA extract. A suitable elution buffer is, for instance, 10 mM Tris-Cl at a pH of 8.5. The magnetic beads may be left in the elution buffer for a certain amount of time in order to allow full retrieval of the DNA fragments from the beads, for instance for a few minutes, e.g. for 10 min. During this time, the mixture may be inverted or stirred. It is also possible to heat the mixture, but room temperature does also work. For example, the mixture may be inverted and then left for 10 min at 20-22 ° C. The magnetic beads may then be removed using the magnet.

The second concentrated DNA extract thus obtained may be used directly in the next steps. Alternatively, it may be further diluted or treated, if desired.

In an embodiment of the present invention, the DNA content of the second concentrated DNA extract is determined by DNA quantification prior to step (f). This allows for a fine-tuning of the amounts and reagents used in the subsequent steps. DNA quantification may be achieved using a Qubit High Sensity Kit (e.g. Qubit dsDNA HS Assay kit, Life technologies; catalog nos Q32851, Q32854) and a Qubit Fluorometer (e.g. from Thermo Fisher Scientific or Invitrogen), but any other suitable devices may also be used.

In step (f) of the method of the present invention, a DNA library is prepared from the second concentrated DNA extract. The DNA library may be prepared using the Field Sequencing Kit or the Rapid Barcoding Kit by Nanopore, for instance. Suitable devices are, e.g. SQK-LRK001 and SQK-RBK004 from Nanopore, which may be used according to the manufacturer's instructions.

In step (g) of the method of the present invention, sequencing of the DNA of the DNA library is performed in order to obtain raw DNA sequences. Sequencing may be achieved using any device and method commonly known in the art.

Steps d) and e) may be optional. In one embodiment, when 16s rRNA gene sequencing approach is used (16s rDNA sequencing), steps d) and e) are not performed and the first concentrate DNA of step c) is used directly for preparing the DNA Library of step f).

In a further embodiment, when using the 16s RNA gene sequencing approach, after extraction and DNA purification DNA barcoding can be used and a barcoded library may be generated. For example the 16S Barcoding Kit (SQK-RAB204) may be used according to the manufacturer's instructions. Other markers that may be used for DNA barcoding of bacteria are the COI, rpoB, cpn60 (Chaperonin 60) or tuf (factor tu) genes.

“DNA barcoding” is a method of sample identification using a synthetic short section of DNA.

Markers used for DNA barcoding are called barcodes. The length of the barcode sequence should be short enough to be used with current sampling source, DNA extraction, amplification and sequencing methods.

The protocol (manufacturer's instructions) of the 16SBarcoding Kit may be modified at the step of purification of the DNA using the purification beads. At this point the elution volume used may be from 10 and 50μl. This increases the DNA quantity and improves the yield of the sequencing run.

In an embodiment of the present invention, the DNA sequencing is performed using MinION Mk1B system by Nanopore (e.g. a flowcell such as FLO-MIN106D, FLO-FLG001). This device is particularly small and light and therefore allows for in field measurements. MinION Mk1B may be used according to the manufacturer's instructions. In another embodiment, flongle loading process can be used according to manufacturer's instructions.

In step (h) of the method of the present invention, human DNA sequences are removed from the raw DNA sequences to obtain non-human DNA sequences. This step allows to ignore DNA stemming from the host (human) and, thus, focus on the microbiota.

It would also be possible to physically remove the human DNA sequences from the DNA samples during sample preparation, e.g. between steps (b) and (c) of the method of the present invention.

However, removing the human DNA sequences “virtually”, i.e. by means of a computer-implemented step, is much easier and faster, thereby reducing the overall time of the method of the present invention. It can also be automated, allowing for use of non-experienced users. Furthermore, removing human DNA sequences in silico avoids introducing a bias: Removing the human DNA sequences physically bears the risk that also non-human DNA sequences are inadvertently removed; and correcting this mistake would require re-doing the whole experiment, which is both costly and time-comsuming. In silico, on the other hand, it is easy to go back to the (complete) raw DNA sequences and repeat the depletion several times, for instance using different genome reference databases, thereby reducing or even fully eliminating the risk of losing non-human DNA sequences.

In an embodiment of the present invention, the raw DNA sequences are aligned on human genome using a pairwise alignment for nucleotide sequences. One suitable genome reference database is Genome Reference Consortium Human Build 38 (GRCh38) from the National Center for Biotechnology Information (NCBI), but other genome reference databases may also be used. The alignment may be done, for instance, using Minimap2, a general-purpose alignment program to map DNA or long mRNA sequences against a large reference database (Heng Li: “Minimap2: pairwise alignment for nucleotide sequences”, Bioinformatics, 34(18), 2018, pages 3094-3100), according to default options.

The non-human DNA sequences thus obtained may be used directly in the further steps. Alternatively, they may also be further filtered in order to simplify taxonomy assignment, etc.

In an embodiment of the present invention, the base pair lengths of the non-human DNA sequences are determined prior to step (i), and only non-human DNA sequences having a base pair length of at least 300 bp, more preferably of at least 400 bp, and most preferably of at least 500 bp, are subjected to the analysis of step (i). This allows for speeding up the taxonomy assignment and improving the precision of the method of the present invention. Short sequences may be removed, for instance, using reformat.sh, an open-source software tool from BBmap (https://github.com/BioInfoTools/BBMap/blob/master/sh/reformat.sh).

In one embodiment Step h) is optional. When 16s RNA gene sequencing approach (16S rDNA sequencing or 16S rRNA gene sequencing) is used, step h) is not performed because the DNA amplified is mainly bacterial DNA.

In step (i) of the method of the present invention, the non-human DNA sequences are analyzed to obtain the microbial profile.

In an embodiment of the present invention, a taxonomy assignment is performed in step (i). The taxonomy assignment may preferably be achieved by aligning the non-human DNA sequences to a non-redundant database covering at least Bacteria, Archaea and Fungi. A suitable program for taxonomy assignment is DIAMOND (B. Buchfink, Xie C., D. Huson: “Fast and sensitive protein alignment using DIAMOND”, Nature Methods 12, 2015, pages 59-60), which may be used according to the default options for long reads.

The different microorganisms (bacteria, fungi, viruses, etc.) can be identified at the different taxonomic levels, including domain, phylum, class, order, genus, and species.

In one embodiment, when barcoded libraries (such as the 16S Barcoding Kit) are used, multiplexing may be used. This is the sequencing in parallel of two or more samples. For example, the base calling software, Guppy (provided by the manufacturer Nanopore technologies), perform a demultiplexing (there is a different file for each sample since each sample has been tagged with a different barcode following the protocol described above). Then, Fastq reads are mapped to a database. In another embodiment multiplexing can be used also for the WGS approach. For example the Rapid barcoding Kit (Rapid Barcoding Kit SQK-RBK004) or Ligation Sequencing Kit (SQK-LSK109) or Ligation Sequencing Kit (SQK-LSK109-XL) from Nanopore can be used.

Then, data obtained using any of the previous described protocols, may be summarized in a table showing the counts and relative abundance for each of the taxonomic ranks (Phylum, Class, Order, Family, Genus, Species) and then can be visualized.

The thus obtained microbial profile may be used for any desired purpose. For instance, the microbial profile may be graphically displayed in order to visualize the composition of the microbiota present on the human's skin.

It may be necessary or advantageous to format and/or further process the microbial profile obtained in step (i) for display. For instance, DIAMOND (see above) allows for various output formats, including BLAST pairwise, tabular and XML, as well as taxonomic classification.

In an embodiment of the present invention, the microbial profile is displayed in a chart. Any chart commonly used for displaying a microbial profile may be used. Interactive charts are particularly advantageous, as they allow a user to focus on particular areas of the charts and reviewing these areas in greater detail.

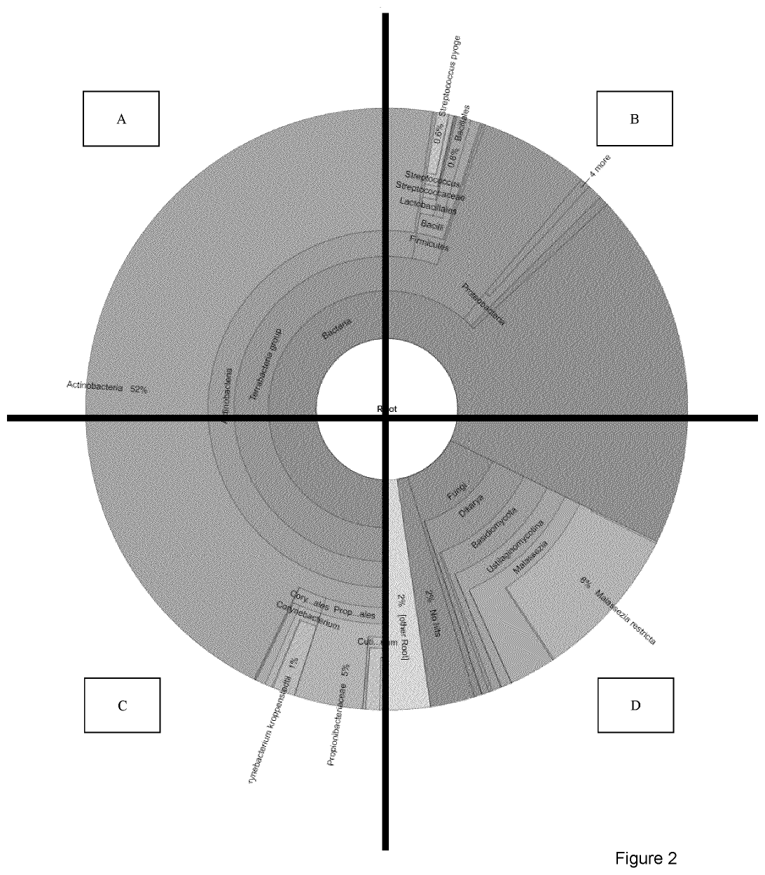

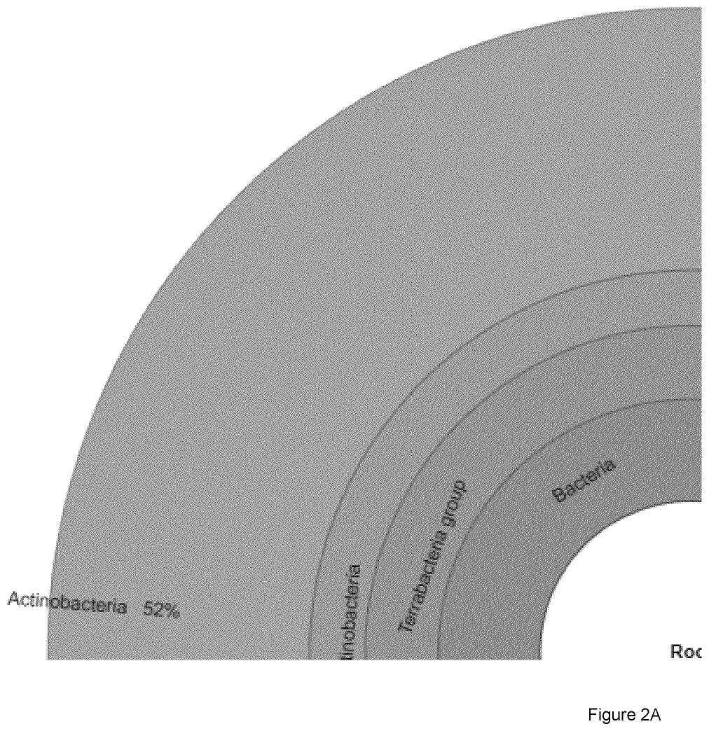

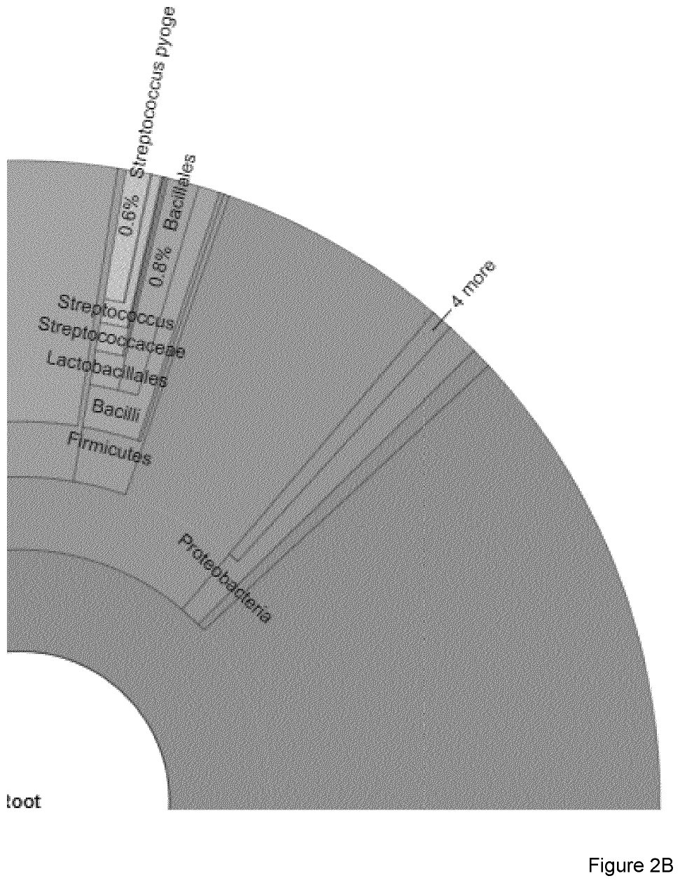

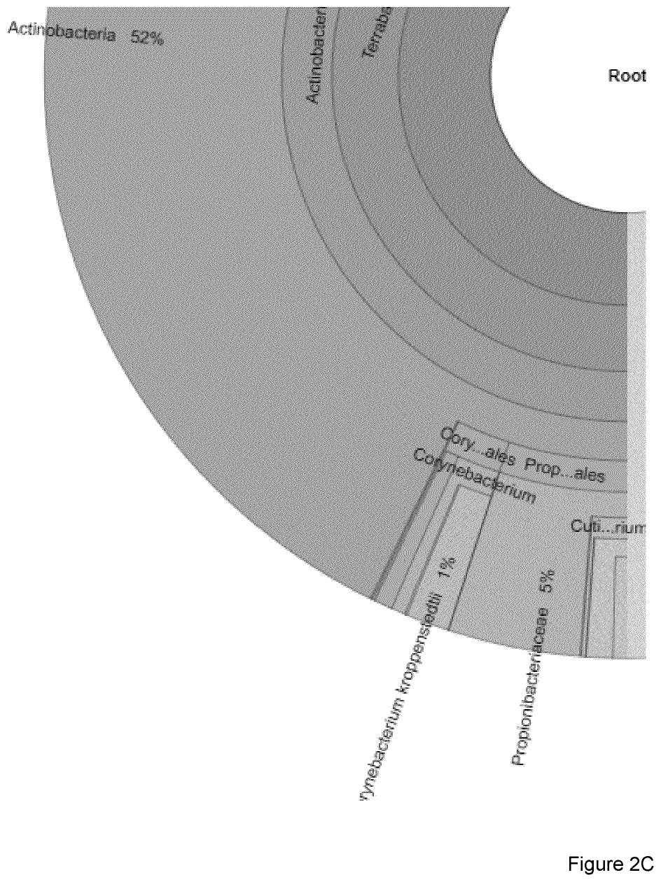

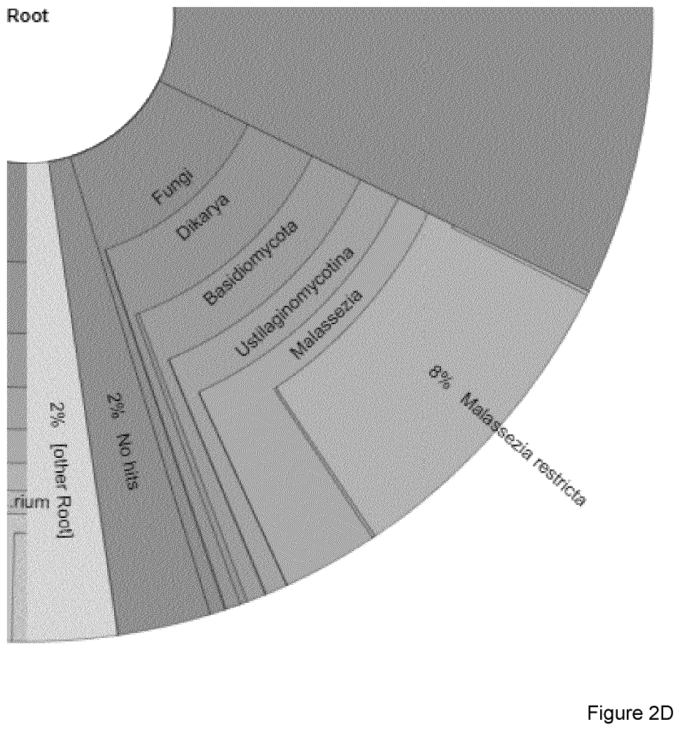

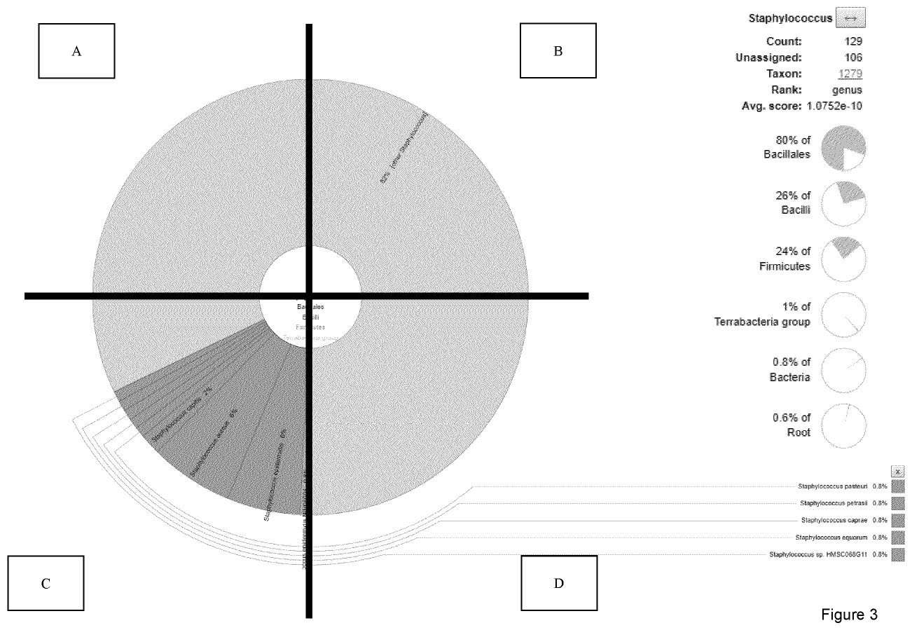

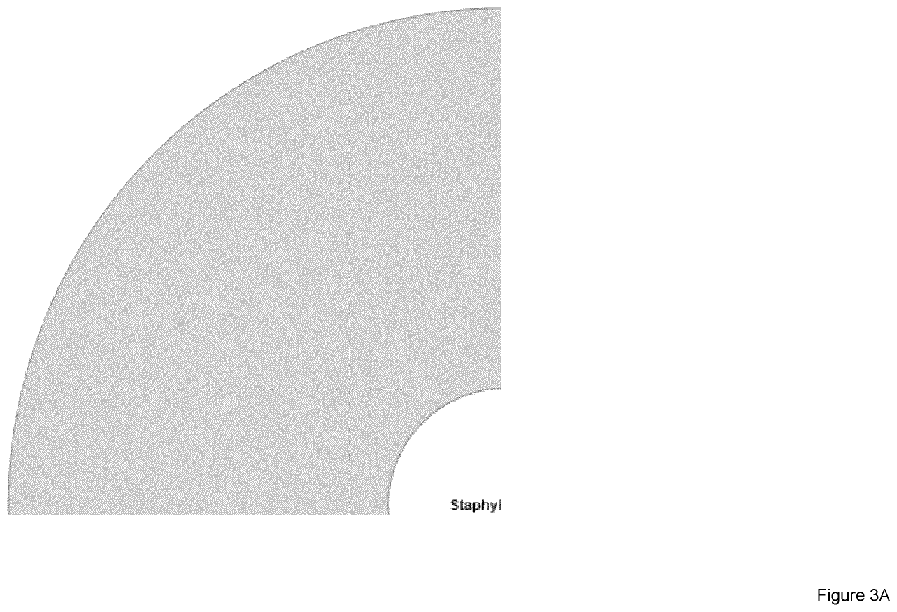

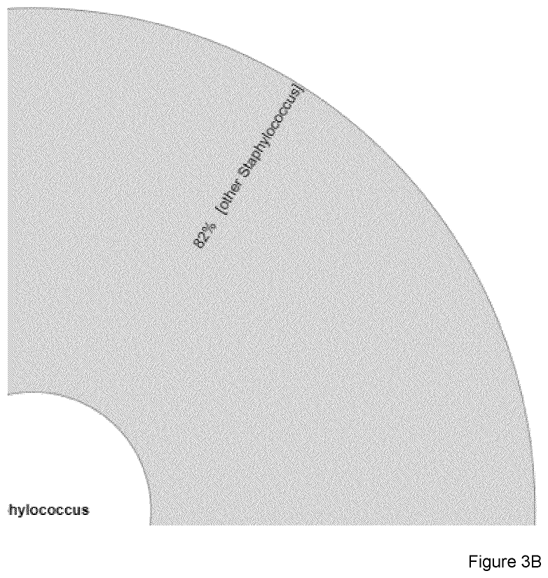

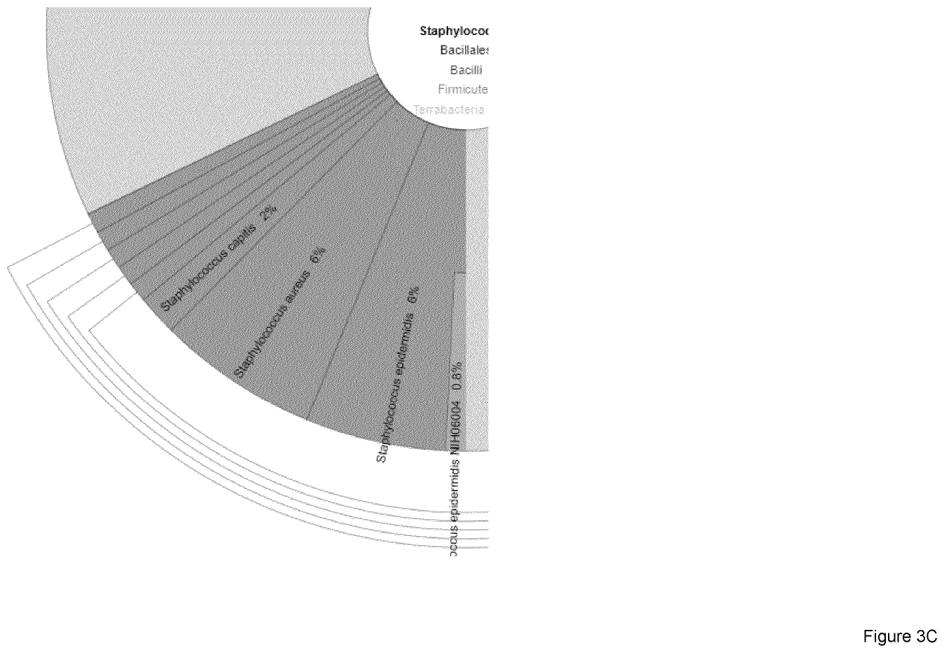

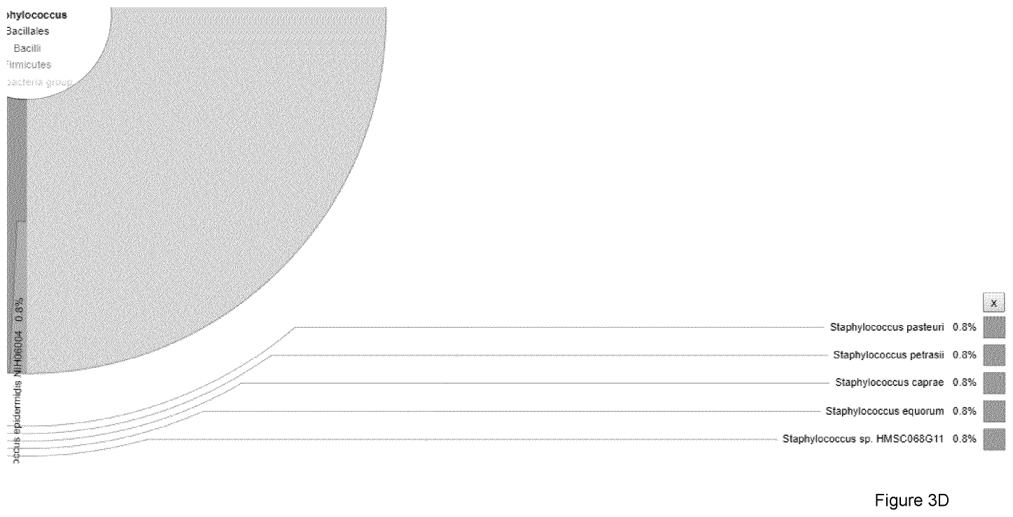

Particularly suitable are interactive pie charts, such as krona (Ondov B D, Bergman N H, and Phillippy A M: “Interactive metagenomic visualization in a Web browser”, BMC Bioinformatics, 2011 Sep 30; 12(1):385; https://Qithub.com/marbl/Krona/wiki). For a krona chart, the output BLAST generated by DIAMOND may be parsed using the default option for GenBank taxonomy. FIGS. 2 and 3 show examples of krona charts, at the domain level (FIG. 2) and the species level (FIG. 3), respectively.

The microbial profile obtained by the method of the present invention may further be used to identify certain qualities of the human skin from which the sample was obtained. There are several studies revealing a relationship between the skin type and the skin microbiota present thereon, for instance on the sebum and hydration level (Mukherjee S, et al. Sci Rep. (2016): “Sebum and Hydration Levels in Specific Regions of Human Face Significantly Predict the Nature and Diversity of Facial Skin Microbiome”), on skin age (Jugé R, et al., J Appl Microbiol 125 (2018): “Shift in skin microbiota of Western European women across aging”), or on aging-related changes in the microbiome (Shibagaki N., et al., Scientific Reports, 7: 10567 | D01:10.1038/s41598-017-10834-9: “Aging-related changes in the diversity of women's skin microbiomes associated with oral bacteria”).

Thus, the method of the present invention does not only provide information on the skin microbiome, such as the bacteria that are present, but also allows for drawing conclusions regarding the quality of the skin, the appearance of the skin, such as looking younger or older, levels of sensitivity, hydration level, or sebum level, among others.

Based on these insights, it is further possible to suggest or provide tailored cosmetic products.

The method of the present invention may be conducted on a single human skin sample. Alternatively, it is also possible to process two or more human skin samples at a time. In this case, certain adaptations to the method of the present invention may be necessary.

To this end, the Rapid Barcoding Kit allows for processing of up to 12 samples simultaneously, for instance (such as the 16S Barcoding Kit (SQK-RAB204) and 16S Barcoding Kit 1-24 (SQK-16S024).

And the DNA library preparation and sequencing steps can be adapted easily. For example, in the DNA library preparation of step (f), tags need to be added on the DNA in order to be able to define which sequences stem from which sample. In addition, after preparation of the raw DNA sequences, an additional de-multiplexing step is necessary, where tags are red and the sequences are assigned to the respective sample. For de-multiplexing, guppy_barcoder from Nanopore may be used.

1. A method for preparing a microbial profile of a human skin sample, comprising the steps of:
(a) extracting DNA from the human skin sample to obtain a crude DNA extract;
(b) treating the crude DNA extract with RNase to remove RNA and obtain an RNA-depleted DNA extract;
(c) purifying the RNA-depleted DNA extract to remove small DNA fragments to obtain a first concentrated DNA extract;
(d) amplifying the DNA of the first concentrated DNA extract to obtain an amplified DNA extract;
(e) purifying the amplified DNA extract to remove small DNA fragments to obtain a second concentrated DNA extract;
(f) preparing a DNA library from the second concentrated DNA extract;
(g) sequencing the DNA of the DNA library to obtain raw DNA sequences;
(h) removing human DNA sequences from the raw DNA sequences to obtain non-human DNA sequences; and
(i) analyzing the non-human DNA sequences to obtain the microbial profile.

2. A method for preparing a microbial profile of a human skin sample, comprising the steps of:
a) extracting DNA from the human skin sample to obtain a crude DNA extract;
b) optionally treating the crude DNA extract with RNase to remove RNA and obtain an RNA-depleted DNA extract;
c) purifying the DNA of step a) or the RNA-depleted DNA extract of step b) to remove small DNA fragments to obtain a first concentrated DNA extract;
d) optionally amplifying the DNA of the first concentrated DNA extract to obtain an amplified DNA extract;
e) optionally purifying the amplified DNA extract of step d) to remove small DNA fragments to obtain a second concentrated DNA extract;
f) preparing a DNA library from the first concentrated DNA extract of step c) or from the second concentrated DNA extract of step e);
g) sequencing the DNA of the DNA library to obtain raw DNA sequences;
h) optionally removing human DNA sequences from the raw DNA sequences to obtain non-human DNA sequences; and
i) analyzing the non-human DNA sequences to obtain the microbial profile.

3. The method according to claim 2, wherein the human skin sample is obtained by swabbing.

4. The method according to claim 2, wherein the DNA extraction of step a) is performed using the DNeasy PowerLyser PowerSoil Kit by Qiagen with a modified procedure, said modified procedure comprising the following steps:
(a1) treating the human skin sample with a PowerBead Solution to obtain a solute sample;
(a2) treating the solute sample with Solution C1, vortexing, and incubating;
(a3) vortexing;
(a4) centrifuging to obtain a first supernatant and a first precipitate;
(a5) mixing Solution C2 with Solution C3 to form a mixture, adding the mixture to the first supernatant, vortexing, and incubating;
(a6) centrifuging to obtain a second supernatant and a second precipitate;
(a7) adding Solution C4 to the second supernatant and vortexing to obtain a treated supernatant;
(a8) loading a first part of the treated supernatant onto an MB Spin Column, centrifuging, and discarding the flow-through;
(a9) loading a second part of the treated supernatant onto the MB Spin Column, centrifuging, and discarding the flow-through;
(a10) loading the remaining treated supernatant onto the MB Spin Column, centrifuging, and discarding the flow-through;
(a11) adding Solution C5 and centrifuging;
(a12) discarding the flow-through and centrifuging the remainder;
(a13) adding Solution C6 to the MB Spin Column; and
(a14) centrifuging and discarding the MB Spin Column to obtain the crude DNA extract.

5. The method according to claim 2, wherein, in step, the crude DNA extract is treated with an aqueous RNAse solution and then incubated.

6. The method according to claim 2, wherein, in step c), the DNA of step a) or the RNA-depleted DNA extract of step b) is purified using magnetic beads, and wherein the purification is a 0.4× purification.

7. The method according to claim 2, wherein, prior to step d), DNA content of the first concentrated DNA extract is determined by DNA quantification.

8. The method according to claim 2, wherein a sequencing technique is selected from the group consisting of whole genome sequencing, 16S rDNA sequencing, and 16S rRNA gene sequencing.

9. The method according to claim 8, wherein the sequencing technique is next generation sequencing or Sanger-sequencing.

10. The method according to claim 2, wherein the DNA amplification of step d) is a Whole Genome Amplification.

11. The method according to claim 2, wherein, in step e), the amplified DNA extract is purified using magnetic beads, and wherein the purification is a 0.4× purification.

12. The method according to claim 2, wherein, prior to step f), DNA content of the second concentrated DNA extract is determined by DNA quantification.

13. The method according to claim 2, wherein, in step f), the DNA library is prepared using a Field Sequencing Kit or a Rapid Barcoding Kit by Nanopore.

14. The method according to claim 2, wherein, in step g), the DNA sequencing is performed using a MinION Mk1B system by Nanopore.

15. The method according to claim 2, wherein, in step h), the raw DNA sequences are aligned on human genome using a pairwise alignment for nucleotide sequences.

16. The method according to claim 2, wherein, prior to step i), the base pair lengths of the non-human DNA sequences are determined and only non-human DNA sequences having a base pair length of at least 300 bp are subjected to the analysis of step i).

17. The method according to claim 2, wherein, in step i), a taxonomy assignment is performed, by aligning to a non-redundant database covering at least bacteria, archaea, and fungi.

18. The method according to claim 2, further comprising the step of:
j) displaying the microbial profile in a chart.

19. The method according to claim 2, wherein in step f) the DNA library is prepared using DNA barcoding.

20. The method according to claim 19, wherein the DNA barcoding is for 16s ribosomal RNA genes, for the COI, rpoB, cpn60 (Chaperonin 60) and/or tuf (factor tu) genes.
